(19) United States
(12) Patent Application Publication
(10) Pub. No.: US 2009/0270787 A1
(43) Pub. Date: Oct. 29, 2009

(54) SYSTEMS AND METHODS FOR CREATING ENLARGED MIGRATION CHANNELS FOR THERAPEUTIC AGENTS WITHIN THE ENDOTHELIUM

(75) Inventors: Randolf von Oepen, Los Altos, CA (US); Travis R. Yribarren, Coarsegold, CA (US); Kevin J. Ehrenreich, San Francisco, CA (US); William E. Webler, JR., San Jose, CA (US); Binh T. Nguyen, Newark, CA (US); Mina Chow, Campbell, CA (US); Gregory W. Chan, San Francisco, CA (US); Rachel Bright, Palo Alto, CA (US)

Correspondence Address: FULWIDER PATTON, LLP (ABBOTT), 6060 CENTER DRIVE, 10TH FLOOR, LOS ANGELES, CA, 90045, US

(73) Assignee: ABBOTT CARDIOVASCULAR SYSTEMS INC., Santa Clara, CA (US)

(21) Appl. No.: 12/108,167

(22) Filed: Apr. 23, 2008

(51) Int. Cl. A61M 25/00

(52) U.S. Cl. 604/20

(57) ABSTRACT

A system for enlarging endothelium migration channels at a treatment site in a coronary vessel wall, to enable enhanced delivery of a therapeutic agent thereto. The system includes an enlarging agent, for enlarging endothelium migration channels at a treatment site in a coronary vessel wall. It also includes a delivery system, for delivering the enlarging agent to the treatment site, so that the enlarging agent will be delivered thereby to enlarge the endothelium migration channels at the treatment site, and for delivering the therapeutic agent to the delivery site. The therapeutic agent will thereby be delivered into the enlarged migration channels at the treatment site, to treat the treatment site with the therapeutic agent.

### BACKGROUND OF THE INVENTION

1. Field of the Invention

This invention is generally related to therapeutic treatment of a coronary vessel wall, and more particularly, to systems and methods for enlarging migration channels within the endothelium for enhanced delivery of therapeutic agents to a treatment site.

2. General Background and State of the Art

Treatment of a coronary vessel wall at a treatment site, for regional therapy of vascular disease, includes delivery of a therapeutic agent into the coronary vessel wall. Delivery of therapeutic agents into the coronary vessel wall relies substantially on diffusion of the therapeutic agents through the endothelium into intercellular gaps. Delivery may occur, for example, by flushing the treatment area with a bolus of agent, or by bringing an agent-loaded surface into contact with the coronary vessel wall.

Delivery of therapeutic agents into the coronary vessel wall may also be accomplished by utilizing drug-eluting stents and balloons, including the deployment of a medical device coated with a therapeutic agent at the treatment site. The therapeutic agent then migrates into the coronary vessel wall to provide the desired benefit.

However, the effective migration of the therapeutic agent into the coronary vessel wall, in healthy and diseased states, is limited by the anatomy of the channels within the endothelium, particularly the size of such channels. Endothelial cell gaps and internal elastic lamina gaps are quite small, and may prevent migration of therapeutic agent particles into the vessel wall, since the gaps are smaller relative to the particles. The small endothelial gaps may also be a problem for delivery of therapeutic agents loaded in polymer particles to create a sustained drug release profile when delivered into the vessel wall.

Therefore, there has been identified a continuing need to provide systems and methods for enhancing the delivery of therapeutic agents to a treatment site in a coronary vessel wall.

### SUMMARY OF THE INVENTION

Briefly, and in general terms, the present invention, in a preferred embodiment, by way of example, is directed to a system for enlarging endothelium migration channels at a treatment site in a coronary vessel wall, to enable enhanced delivery of a therapeutic agent thereto. The system includes an enlarging agent, for enlarging endothelium migration channels at a treatment site in a coronary vessel wall. It further includes a delivery system, for delivering the enlarging agent to the treatment site, so that the enlarging agent will be delivered thereby to enlarge the endothelium migration channels at the treatment site, and for delivering the therapeutic agent to the delivery site, so that the therapeutic agent will be delivered thereby into the enlarged migration channels at the treatment site, to treat the treatment site with the therapeutic agent.

In accordance with other aspects of the invention, there is further provided an enlarging agent which comprises an etching agent, transmissible through the delivery system to the treatment site, wherein the etching agent is acidic, and is able to etch the endothelium for enlarged migration channels at the treatment site.

In other aspects of the invention, the enlarging agent comprises a hydrophilic agent, transmissible through the delivery system to the treatment site, wherein the hydrophilic agent is able to attract water from the surrounding endothelial cells, resulting in a decrease in volume occupied by the surrounding endothelial cells and an increase in the volume of the endothelial cell gaps, for enlarged migration channels.

In another yet other aspects of the invention, the enlarging agent comprises radiant energy, and the system further includes a radiant energy element which comprises a source of radiant energy, wherein the endothelium includes endothelial cells and endothelial cell gaps, and the radiant energy element is able to increase the volume of the endothelial cell gaps to enlarge the migration channels.

In another aspect of the invention, the system further includes a closure agent to stimulate the vessel wall after delivery of the therapeutic agent to close the migration channels and trap the therapeutic agent within the vessel wall.

In still other aspects of the invention, there is further provided a delivery system which includes a catheter. The catheter includes an expandable balloon structure, positioned relative to the catheter distal end and associated with the catheter body. The catheter further includes an inflation lumen, the expandable balloon structure includes a porous surface, and the enlarging agent is transmissible to the treatment site through the inflation lumen and the porous surface of the expandable balloon structure.

These and other aspects and advantages of the invention will become apparent from the following detailed description and the accompanying drawings, which illustrate by way of example the features of the invention.

### BRIEF DESCRIPTION OF THE DRAWINGS

FIG. 1 is an elevational view, partly exploded and partly in cross-section, of an enlarging system embodying features of the present invention, wherein the delivery system and the enlarging agent are disposed within a vessel at a treatment site;

FIG. 2 is an elevational view, partly fragmentary and partly in cross-section, similar to that shown in FIG. 1, wherein the delivery system is in its expanded position within the vessel at the treatment site;

FIG. 3 is an elevational view, partly fragmentary and partly in cross-section, similar to that shown in FIG. 2, wherein the enlarging agent is transmitted through the expanded delivery system within the vessel at the treatment site;

FIG. 4 is an elevational fragmentary view, partly in cross-section, of enlarged migration channels formed by transmission of the enlarging agent through the delivery system in the vessel at the treatment site, similar to that shown in FIG. 3;

FIG. 5 is an elevational view of a delivery system including a radiant energy source connected thereto and radiant energy transmitted therethrough;

FIG. 6 is an elevational partly-fragmentary view of the delivery system and the radiant energy enlarging agent transmitted therethrough;

FIG. 7 is an elevational cross-sectional view, taken on line 7-7 in FIG. 5, of the delivery system and the radiant energy enlarging agent transmitted therethrough;

FIG. 8 is an elevational partly-fragmentary partly cross-sectional view of a delivery system including a sheath, and a radiant energy enlarging agent and a therapeutic agent transmitted therethrough, in the vessel at the treatment site;

FIG. 9 is an elevational partly-fragmentary partly cross-sectional view of a delivery system including a sheath and an expandable balloon structure for transmission of a radiant energy enlarging agent therethrough;

FIG. 10 is an elevational view, partly fragmentary and partly in cross-section, of a delivery system including an expanded balloon and a radiant energy enlarging agent transmitted therethrough in a vessel at the treatment site;

FIG. 11 is an elevational cross-sectional view, taken along line 11-11 in FIG. 10, of the delivery system for delivery of the enlarging agent;

FIG. 12 is an elevational view of a delivery system including an expandable member for transmitting an enlarging agent and a sealing agent therethrough;

FIG. 13 is a cross-sectional view, taken along line 13-13 of FIG. 12, of the delivery system;

FIG. 14 is a cross-sectional view, taken along line 14-14 of FIG. 12, of the expanding member in the delivery system; and

FIG. 15 is a perspective partly-broken view of a delivery system including an expandable member for transmitting an enlarging agent and a sealing agent, similar to FIG. 12.

### DETAILED DESCRIPTION OF THE PREFERRED EMBODIMENTS

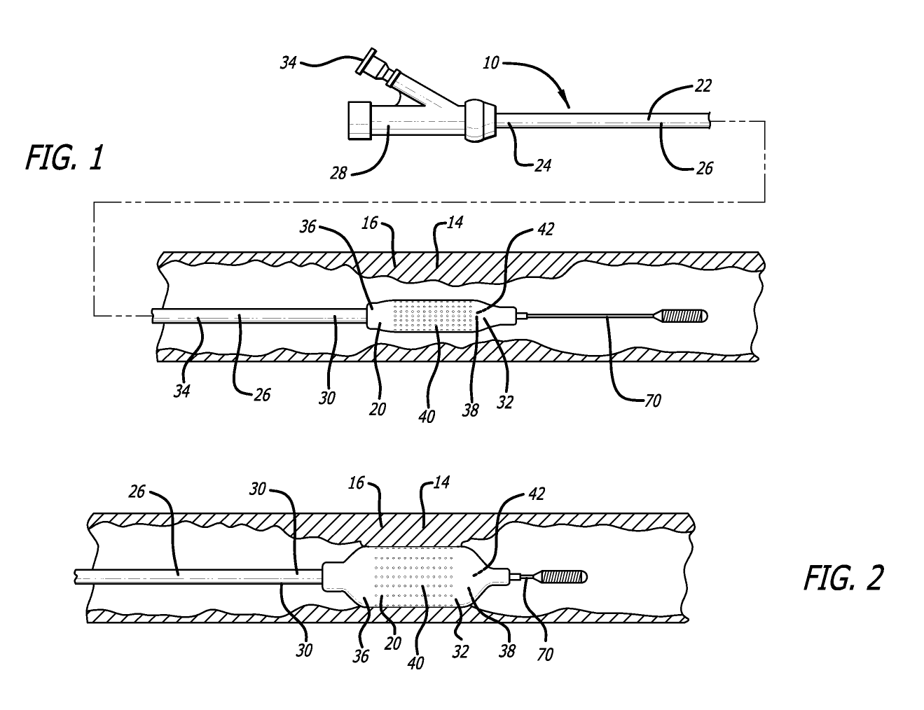

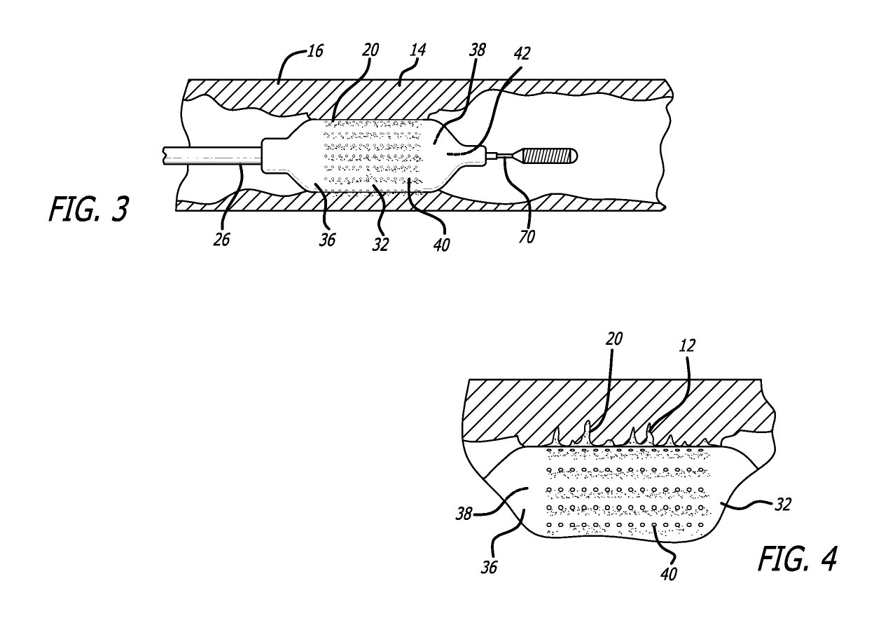

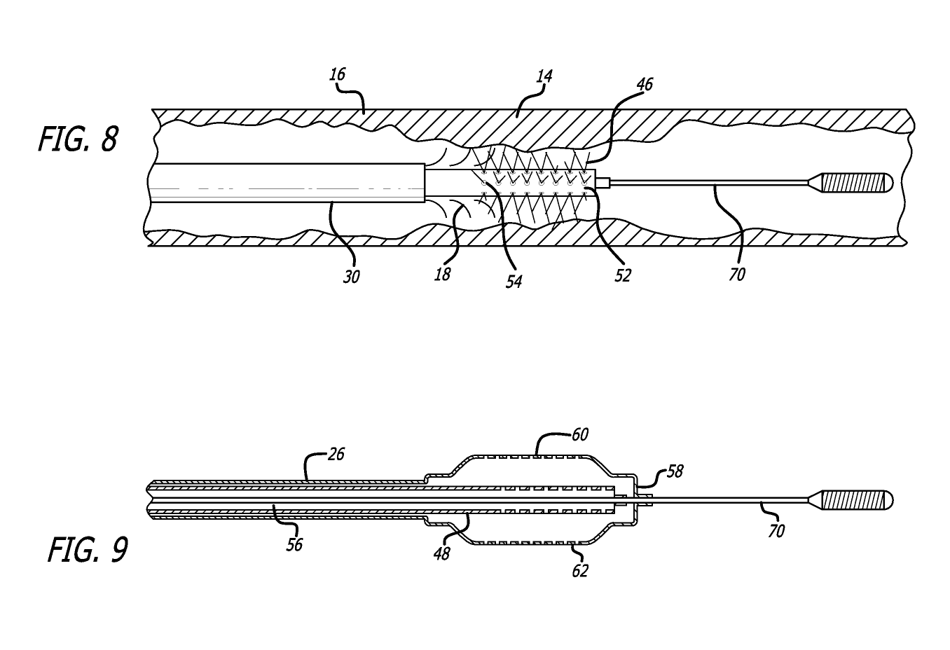

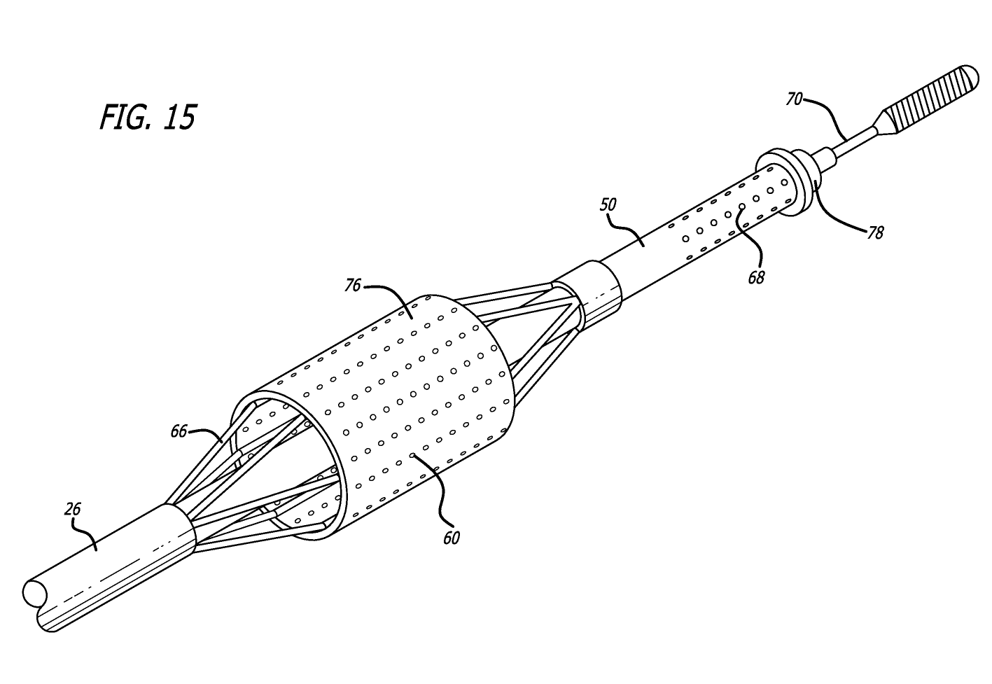

Referring to the drawings, FIGS. 1-15, in which like reference numerals refer to like or corresponding parts, the system 10 according to the invention enables the enlarging of endothelium migration channels 12 at a treatment site 14 in a coronary vessel wall 16, so as to enable enhanced delivery of a therapeutic agent 18 (FIG. 8) thereto for regional therapy of vascular disease. The therapeutic agent 18 may comprise an anti-proliferative, an anti-inflammatory, and/or an anti-lipid drug. Other therapeutic agents that may be utilized include antineoplastic, antiplatelet, anti-coagulant, anti-fibrin, antithrombotic, antimitotic, antibiotic, antiallergic and antioxidant compounds. The system 10 creates larger migration channels 12 within the endothelium, for creating improved pathways for delivery of the therapeutic agents 18. The system 10 includes an enlarging agent 20, for enlarging endothelium migration channels 12 at a treatment site 14 in a coronary vessel wall 16, and a delivery system 22, for delivering the enlarging agent 20 to the treatment site 14. The enlarging agent 20 is delivered by the system 10 to enlarge the endothelium migration channels 12 at the treatment site 14, and the system 10 delivers the therapeutic agent 18 to the treatment site 14 so that the therapeutic agent 18 will be delivered thereby into the enlarged migration channels 12 at the treatment site 14, to treat the treatment site 14 with the therapeutic agent 18.

FIG. 1 presents a first embodiment of a system 10 wherein the delivery system 22 includes a catheter 24. The catheter 24 includes a body 26 which includes a proximal end 28 and a distal end 30. The catheter 24 further includes an expandable balloon structure 32, positioned relative to the catheter distal end 30, associated with the catheter body 26. The catheter 24 also includes an inflation lumen 34, extending substantially the length of the catheter body 26 with which the expandable balloon structure 32 is in fluid communication. The inflation lumen 34 enables fluids to be moved through the catheter body 26 to inflate and deflate the expandable balloon structure 32. The expandable balloon structure 32 includes an external surface 36 and an internal surface 38, and comprises a porous surface 40, whereby the external surface 36 is in fluid communication with the internal surface 38. The enlarging agent 20 is transmissible through the inflation lumen 34 and the porous surface 40 of the expandable balloon structure 32. The porous surface 40 of the expandable balloon structure 32 enables fluid to flow therethrough during inflation and after a certain pressure is reached. The certain pressure for fluid flow through the porous surface 40 of the expandable balloon structure 32 may be in the range of 0.1 to 1.0 atmospheres. The system 10 may also include a guidewire lumen 42 extending substantially the length of the catheter body 26.

The enlarging agent 20 may comprise an etching agent, transmissible through the inflation lumen 34 and the porous surface 40 of the expandable balloon structure 32 of the delivery system 22 to the treatment site 14. The etching agent 20 may be acidic, and is able to etch the endothelium.

Referring to FIG. 2-4, a method is shown for utilizing the system 10 to enlarge endothelium migration channels 12 at the treatment site 14 in the coronary vessel wall 16, to enable enhanced delivery of the therapeutic agent 18 thereto. The method comprises advancing the enlarging agent 20 through the delivery system 22 to the treatment site 14 in the coronary vessel wall 16, so as to enlarge the endothelium migration channels 12 at the treatment site 14, and moving the therapeutic agent 18 into the enlarged migration channels 12 at the treatment site 14, so as to treat the treatment site 14 with the therapeutic agent 18. The method includes tracking the system 10 over the guidewire lumen 42 through the vasculature 16 to the treatment site 14. Once the system 10 is in place, the expandable balloon structure 32 is inflated by injecting either the etching agent 20, or another inflation fluid such as saline, through the inflation lumen 34, thereby inflating the expandable balloon structure 32 radially outwardly, bringing the porous surface 40 of the expandable balloon structure 32 into contact with the vessel wall 16, and inflating the expandable balloon structure 32 by advancing the inflation medium through the inflation lumen 34, inflating the expandable balloon structure 32 so as to contact the adjacent vessel wall 16. The method further comprises advancing the etching agent 20 through the inflation lumen 34 into the expandable balloon structure 32 and out of the porous surface 40 thereof, enabling the etching agent 20 to etch the endothelium in the localized areas of the treatment site 14, and creating channels 12 thereby into the vessel wall 16. The method also includes administering a solution containing the therapeutic agents 18 through the expandable balloon structure 32 into the created channels 12, and trapping the therapeutic agents 18 within the coronary vessel wall 16, for treating the vascular wall 16.

In an alternative embodiment, the enlarging agent 20 comprises a hydrophilic agent, transmissible through the inflation lumen 34 and the porous surface 40 of the expandable balloon structure 32 of the delivery system 22 to the treatment site 14 and into the vessel wall 16. The hydrophilic agent may comprise hyaluronic acid. The endothelium includes endothelial cells and endothelial cell gaps, and water in the endothelial cells surrounds the endothelial cell gaps. The hydrophilic agent is able to enter the endothelial cell gaps, and attract water from the surrounding endothelial cells, resulting in a decrease in volume occupied by the surrounding endothelial cells and an increase in the endothelial cell gaps. The hydrophilic agent is able to quickly dissolve along with the surrounding endothelial cell water, creating enlarged channels 12 for entry of the therapeutic agent 18 into the vessel wall 16. The therapeutic agent 18 is then introduced through the delivery system 22 into the coronary vessel wall 16. The endothelial cells rehydrate, further closing the endothelial gaps, and trapping the therapeutic agent 18, enabling treatment of the vascular disease at the treatment site 14.

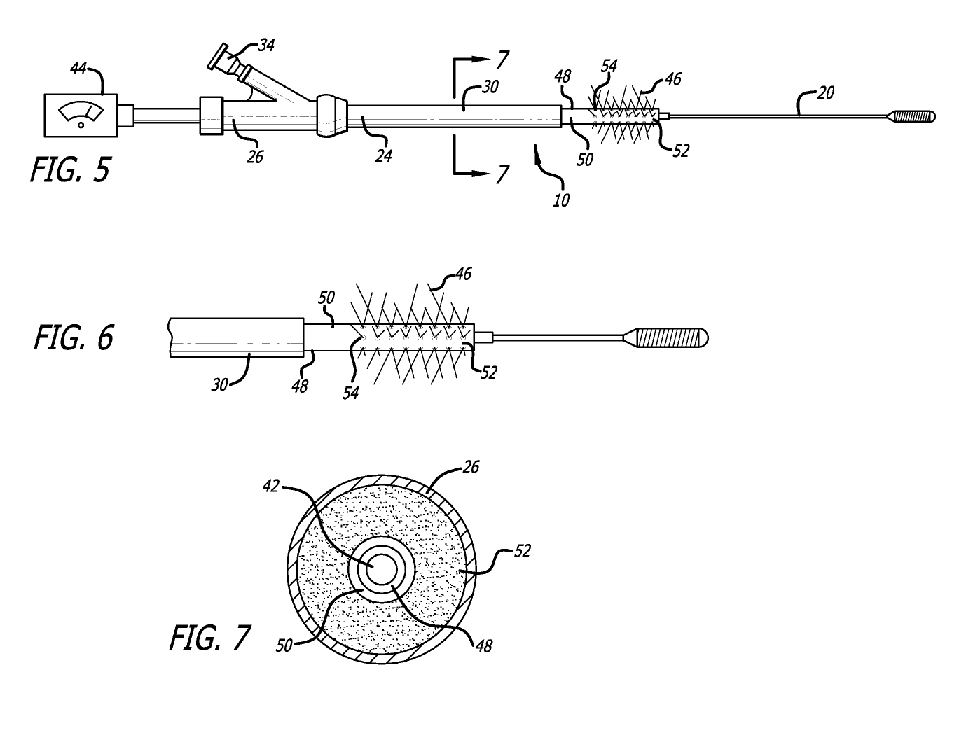

In a further embodiment as illustrated in FIGS. 5-7, the enlarging agent 20 is energy based, and may be utilized with the elongated body 26 of the catheter 24, wherein a radiant energy source 44 is connectable to the catheter body 26, and which can deliver the enlarging agent 20, which may comprise radiant energy 46, through the length and to the distal end of the catheter body 26, to a radiant energy element 48. The endothelium includes endothelial cells and endothelial cell gaps which comprise internal elastic lamina gaps, and the radiant energy element 48 is able to increase the volume of the internal elastic lamina gaps. The radiant energy source 44 may be attached near the proximal end 28 of the catheter body 26, which transmits the radiant energy 46 towards the distal end 30 thereof. The radiant energy 46 of the radiant energy element 48 may comprise laser energy, ultra-violet light energy, radio frequency energy, short pulsed ultraviolet laser, or excimer laser as a radiant energy source. The radiant energy element 48 includes a sheath 50 which is able to transmit the radiant energy 46 over the length thereof. The sheath 50 includes a distal end 52, and, near the distal end 52, the sheath 50 may include discontinuities 54 such as holes, which enable the radiant energy 46 to escape into the surrounding space.

The radiant energy element 48 may comprise one or more fiber optic light guides capable of delivering light. Fiber optic light guides may be formed from glass or polymer and may be sized at about 12 micrometers. A cladding or reflective jacketing may be disposed over the proximal section of the light guides to prevent light loss and improve transmission efficiency.

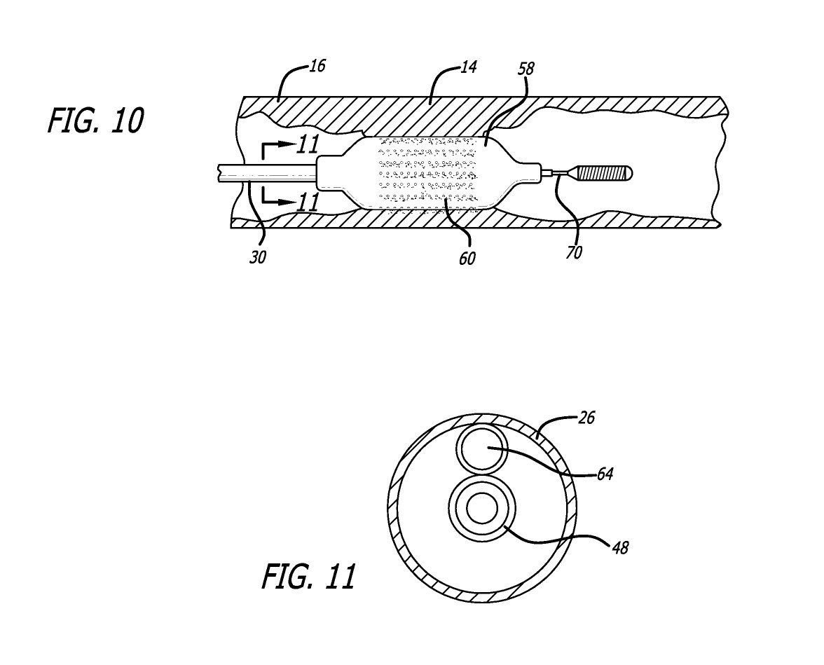

The system 10 may further include an intermediate fluid lumen 56, between the catheter body 26 and the energy element 48, able to deliver the therapeutic agent 18 into the coronary vessel 16 relative to the distal end 52 of the energy element 48. An exit port for a guidewire 20 may be located in the catheter 24. Alternatively, the radiant energy element 44 may be attached to an additional port, and the guidewire 20 may exit where the radiant energy element 44 is, as shown attached in FIG. 5. There is a connector 34 for delivering fluid through the intermediate fluid lumen 56. There is a space between the radiant energy element 48 and the guidewire 70, as seen in FIGS. 7 and 11, to prevent slideable contact therebetween.

A method of using the system 10 includes tracking the system 10 through the vasculature to the treatment site 14, as seen in FIG. 8. Upon positioning the system 10 in place at the treatment site 14, the system 10 is coupled to the radiant energy source 44, and the radiant energy element 48 is activated to transmit the radiant energy 46 therethrough. The radiant energy 46 escapes through the discontinuities 54 in the sheath 50 and is directed toward the surrounding vessel wall 16. The discontinuities 54 are sized and patterned to create flux areas 12 at the vessel wall 16 that are consistent with the channel sizes for delivery of the therapeutic agent 18. The radiant energy 46 delivered to the flux areas 12 causes ablation of the endothelium, continuing to a specified depth into or through the internal elastic lamina depending upon the density and time of exposure of the radiant energy supplied. Following ablation of the vessel wall 16, the therapeutic agent 18 is delivered through the catheter body 26 into the coronary vessel, causing the therapeutic agent 18 to migrate into the ablated channels 12, delivering the therapeutic agent 18 to the treatment site 14.

Alternatively, as illustrated in FIG. 9, the delivery system 22 also includes an expandable member 58 positionable relative to the catheter distal end 30. The expandable member 58 may be a porous balloon, wherein the pores 60 serve several functions. The pores 60 may replace or complement the discontinuities 54 in the sheath 50. For this purpose, the balloon 58 may be comprised of material which may be opaque or have a thickness or composition that may inhibit transmission of the radiant energy 46. Further, the pores 60 may allow localized delivery of the therapeutic agent 18 to the areas of ablation. The radiant energy element 48 and the expandable member 58 are shown as interconnected for support of the inflation lumens 34.

FIGS. 10-11 present a further method of using the system 10, which includes tracking the system 10 to the treatment site 14. When the system 10 is in place, the balloon 58 is deployed, and the balloon surface 62 is brought into contact with the vessel wall 16. The system 10 is coupled to the radiant energy source 44, and the radiant energy element 48 is activated to transmit the radiant energy 46 therethrough. The radiant energy 46 escapes from the radiant energy element 48 through the balloon pores 60 and is directed toward the surrounding vessel wall 16. The discontinuities 54 are sized and patterned to create flux areas 12 at the vessel wall 16 that are consistent with the channel sizes for delivery of the therapeutic agent 18. The radiant energy 46 is delivered to the flux areas 12, causing ablation of the endothelium, continuing to a specified depth into or through the internal elastic lamina depending upon the radiant energy supplied. Following ablation of the vessel wall 16, the therapeutic agent 18 is delivered through the catheter body 26 and the balloon 58 into the coronary vessel 16, causing the therapeutic agent 18 to migrate into the ablated channels 12, delivering the therapeutic agent 18 to the treatment site 14.

In another embodiment, the radiant energy 46 of the radiant energy element 48 may comprise light energy, and the radiant energy element 48 may include an optical fiber for delivering the light energy relative to the distal end 30 of the catheter 22. The radiant energy 46 escapes through the balloon pores 60 and is diffused and directed toward the surrounding vessel wall 16. The balloon 60 includes a diameter, and a surface material on the diameter, for diffusing the radiant energy 46. The surface material may comprise a reflective surface material, such as a metalized surface material. The balloon 58 may include an optical diffusive device for diffusing the radiant energy 46 toward the surrounding vessel wall 16. The optical surface device may include a reflector. The optical surface device may include a scored outer fiber optic layer, having discontinuities 54 at intermittent locations. The metalized balloon surface may include discontinuities 54 such as gaps or holes which enable the radiant energy 46 to escape and be directed toward the surrounding vessel wall 16.

Alternatively, the expandable member 58 may be able to create ablated channels 12, while the therapeutic agent 18 may be able to be delivered other than through the pores 60. The expandable member 58 may be able to be sized to displace blood within the treatment area 14, without circumferentially contacting the vessel wall 16. The therapeutic agent 18 may be delivered by a device other than the expandable member 58, since the expandable member 58 may be used to create ablated channels 12, and the therapeutic agent 18 may be delivered by a subsequent device after channel ablation. The therapeutic agent 18 also may be delivered by flushing the treatment site 14 therewith after channel ablation.

In another embodiment, the delivery system 22 may include an expandable member 58 which comprises a metallic cage, which is able to expand by retraction of the sheath 50, and is collapsed by advancement of the sheath 50, and which is able to create channels 12 and apply the treatment agent. The metallic cage may be comprised of nitinol. The metallic cage may include a membrane for covering thereof, able to apply the therapeutic agent 18 without unduly restricting vessel blood flow. The system 10 may further include an optical fiber positioned on the expandable member 58. The optical fiber may include scores therein, such that the radiant energy 46 escapes through the scores and is directed towards the surrounding vessel wall 16, such that the creation of channels 12 may be accomplished with the same device that applies the treatment agent 18.

Following delivery of the therapeutic agent 18, a closure agent such as a stimulus may be provided to the vessel wall 16 to urge the closure of the migration channels 12. The closure agent may comprise a vasoconstrictive agent which causes the migration channels 12 to restrict, thereby trapping the therapeutic agent 18 within the vessel wall 16, providing the clinical benefit of potentially lower drug doses. Alternatively, the radiant energy 46 may be transmissible through the system 10 that promotes restriction of the vessel wall 16, thereby urging closure of the migration channels 12.

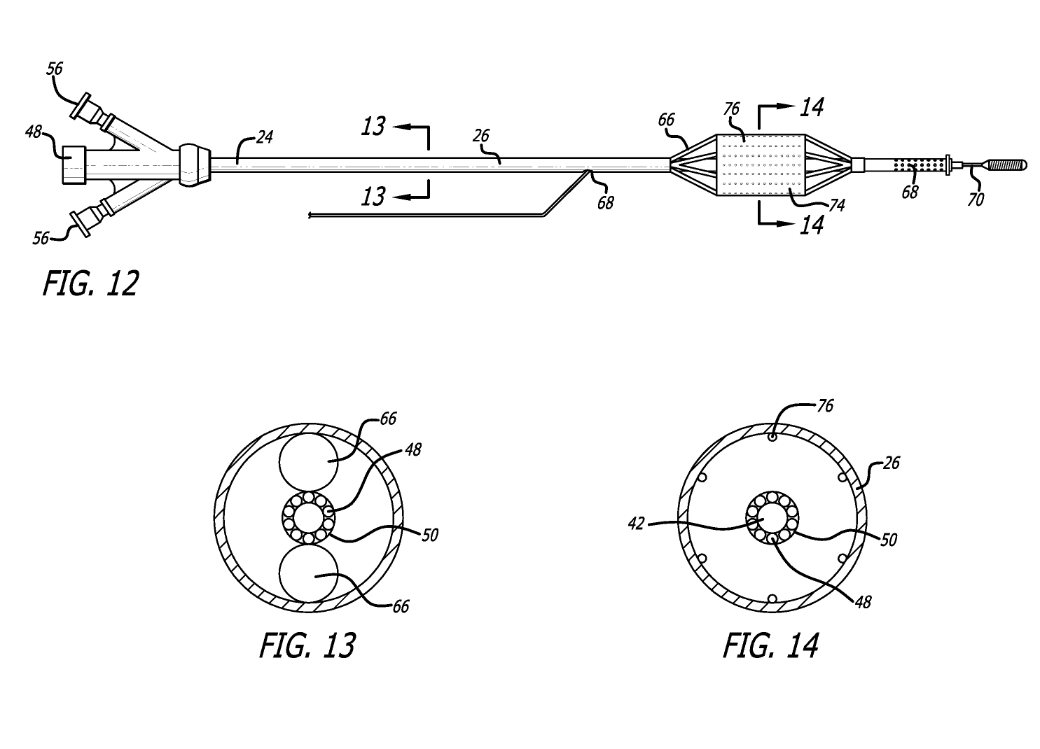

In an alternative embodiment, as shown in FIGS. 12-15, a system 10 further comprises a sealing agent to seal the ablated channels 12 in the vessel wall 16 after delivery of the therapeutic agent 18. The sealing agent may comprise a photo-curable agent. The photo-curable agent may comprise a photo-cure cyanocrylate adhesive. The radiant energy source 44 is able to be reactivated after the sealing agent to deliver the energy necessary to activate the adhesive. The radiant energy element 48 may comprise fiber bundles, as in FIGS. 13 and 14, or hollow light guides, as in FIGS. 7 and 11. The catheter body 26 may include a dedicated channel 64, as seen in FIG. 11, for delivery of the photo-activated agent. This may prevent dilution of the agent, and the difficulties that may occur otherwise, if the various agents had to be aspirated and exchanged in order to deliver them to the treatment site 16.

The system 10 may be axially movable. Therapeutic agent lumens 66 may be connected at the proximal end 28 of the catheter 24. A port 68 in the catheter body 26 may be provided for extension of a guidewire 70 therethrough. The expandable member 58 may comprise an expandable reservoir which may be provided for the therapeutic agent 18. The expandable reservoir 58 includes a proximal end 72 and a distal end 74 and compartments therein. Expandable support members 76 such as struts may be connected to the catheter body 26 and the proximal end 72 and the distal end 74 of the expandable reservoir 58, for enabling expansion thereof. The support members 76 may comprise tubes for supplying the sealant agent and for providing support. A stop 78 may be able to be pulled until it stops at the distal end 74 of the expandable reservoir 58.

When tracking the energy element 46 into the expandable member 58 which may comprise a cage, the stop 78 may be pulled until it stops at the distal end 74 of the cage 58. The cage 58 may be pushed forward with the catheter shaft 24 to deploy the delivery element 22 against the vessel lumen 16. The therapeutic agent 18 may be infused from the cage reservoir 58 out through an infusion membrane. After infusion of the therapeutic agent 18, the sealant agent may be infused from the support channels. The radiant energy source is able to be re-activated after the sealing agent to deliver the energy necessary to activate the sealing agent.

While the particular systems and methods as shown and disclosed in detail herein are fully capable of obtaining the objects and providing the advantages previously stated, it is to be understood that they are merely illustrative of the presently preferred embodiment of the invention, and that no limitations are intended to the details of construction or design shown herein other than as described in the appended claims.

1. A system for enlarging endothelium migration channels at a treatment site in a coronary vessel wall, to enable enhanced delivery of a therapeutic agent thereto, comprising:
an enlarging agent, for enlarging endothelium migration channels at a treatment site in a coronary vessel wall; and
a delivery system, for delivering the enlarging agent to the treatment site, so that the enlarging agent will be delivered thereby to enlarge the endothelium migration channels at the treatment site, and for delivering the therapeutic agent to the delivery site, so that the therapeutic agent will be delivered thereby into the enlarged migration channels at the treatment site, to treat the treatment site with the therapeutic agent.

2. A system as in claim 1, further comprising a closure agent to stimulate the vessel wall after delivery of the therapeutic agent to close the migration channels and trap the therapeutic agent within the vessel wall.

3. A system as in claim 1, wherein the enlarging agent comprises an etching agent, transmissible through the delivery system to the treatment site.

4. A system as in claim 1, wherein the enlarging agent comprises a hydrophilic agent, transmissible through the delivery system to the treatment site.

5. A system as in claim 1, wherein the enlarging agent comprises a radiant energy element which comprises a source of radiant energy.

6. A system as in claim 1, wherein the delivery system includes a catheter.

7. A system as in claim 1, further comprising a closure agent to stimulate the vessel wall after delivery of the therapeutic agent to close the migration channels and track the therapeutic agent within the vessel wall.

8. A system as in claim 3, wherein the etching agent is acidic, and is able to etch the endothelium.

9. A system as in claim 4, wherein the hydrophilic agent comprises hyaluronic acid.

10. A system as in claim 4, wherein the endothelium includes endothelial cells and endothelial cell gaps, and water in the endothelial cells surrounding the endothelial cell gaps, and the hydrophilic agent is able to enter the endothelial cell gaps, and attract water from the surrounding endothelial cells, resulting in a decrease in volume occupied by the surrounding endothelial cells and an increase in the endothelial cell gaps.

11. A system as in claim 5, wherein the endothelium includes endothelial cells and endothelial cell gaps which comprise internal elastic lamina gaps, and the radiant energy element is able to increase the endothelial cell gaps.

12. A system as in claim 5, wherein the radiant energy of the radiant energy comprises laser energy.

13. A system as in claim 5, wherein the radiant energy of the radiant energy element comprises ultra-violet light energy.

14. A system as in claim 5, wherein the radiant energy of the radiant energy element comprises radio frequency energy.

15. A system as in claim 5, wherein the radiant energy of the radiant energy element comprises short-pulsed ultraviolet laser.

16. A system as in claim 5, wherein the radiant energy of the radiant energy element comprises excimer laser.

17. A system as in claim 5, wherein the radiant energy element includes a sheath which is able to transmit radiant energy over the length thereof.

18. A system as in claim 5, wherein the radiant energy element includes a plurality of discontinuities near the distal end thereof, which enable radiant energy to escape into the surrounding space.

19. A system as in claim 5, wherein the delivery system includes a catheter.

20. A system as in claim 5, wherein the delivery system includes a catheter, the radiant energy of the radiant energy element comprises light energy, and the radiant energy element includes an optical fiber for delivering the light energy relative to the distal end of the catheter.

21. A system as in claim 6, wherein the catheter includes a body which includes a proximal end and a distal end.

22. A system as in claim 6, wherein the closure agent comprises a vasoconstrictive agent which causes the migration channels to restrict.

23. A system as in clam 10, wherein the hydrophilic agent is able to quickly dissolve along with the surrounding endothelial cell water, creating enlarged channels for entry of the therapeutic agent into the vessel wall.

24. A system as in claim 17, wherein the delivery system includes an extendable member which comprises a metallic cage, which is able to expand by retraction of the sheath, and is collapsed by advancement of the sheath, and which is able to create channels and apply the treatment agent.

25. A system as in claim 18, wherein the sheath discontinuities comprise holes.

26. A system as in claim 18, wherein the sheath discontinuities are sized and patterned to create flux areas at the vessel wall that are consistent with optimal channel sizes for delivery of the therapeutic agent.

27. A system as in claim 19, wherein the catheter includes a body which includes a proximal and a distal end.

28. A system as in claim 20, wherein the catheter further includes an expandable balloon structure, which includes a porous surface, such that the radiant energy escapes through the balloon pores and is diffused and directed toward the surrounding vessel wall.

29. A system as in claim 21, wherein the catheter further includes an expandable member, positioned relative to the catheter distal end, associated with the catheter body.

30. A system as in claim 21, wherein the catheter further includes an expandable balloon structure, positioned relative to the catheter distal end, associated with the catheter body.

31. A system as in claim 21, wherein the catheter further comprises a guidewire lumen, extending substantially the length of the catheter body.

32. A system as in claim 22, wherein the closure agent comprises radiant energy, which is transmissible through the delivery system, and which promotes restriction of the vessel wall.

33. A system as in claim 23, wherein the therapeutic agent is introduced through the delivery system into the coronary vessel wall.

34. A system as in claim 24, wherein the metallic cage is comprised of NITINOL.

35. A system as in claim 27, wherein the catheter body is elongated.

36. A system as in claim 27, wherein the radiant energy element is able to transmit and deliver radiant energy through the length of the catheter body and to the distal end thereof.

37. A system as in claim 27, wherein the radiant energy element is able to be attached to the catheter body relative to the proximal end thereof.

38. A system as in claim 27, further including an intermediate lumen, between the catheter body and the energy element, able to deliver the therapeutic agent into the coronary vessel relative to the distal end of the energy element.

39. A system as in claim 27, wherein the catheter further comprises a guidewire lumen, extending substantially the length of the catheter body.

40. A method of enlarging endothelium migration channels at a treatment site in a coronary vessel wall, to enable enhanced delivery of a therapeutic agent thereto, in a system which comprises an enlarging agent, for enlarging endothelium migration channels at a treatment site in a coronary vessel wall, and a delivery system, for delivering the enlarging agent to the treatment site, so that the enlarging agent will be delivered thereby to enlarge the endothelium migration channels upon delivery of the enlarging agent to the treatment site, wherein the method comprises:
delivering the enlarging agent through the delivery system to the treatment site in the coronary vessel wall; and
enlarging endothelium migration channels by the enlarging agent upon delivery of the enlarging agent to the treatment site.

41. A method as in claim 40, further comprising a closure agent to stimulate the vessel wall after delivery of the therapeutic agent to close the migration channels and trap the therapeutic agent within the vessel wall, and wherein the method further comprises stimulating the vessel wall with the closure agent after delivery of the therapeutic agent, to close the migration channels and trap the therapeutic agent within the vessel wall.

42. A method as in claim 40, wherein the delivery system comprises a catheter, and the catheter includes a body and a guidewire lumen, extending substantially the length of the catheter body, and wherein the method further includes tracking the catheter over the guidewire through the vasculature to the treatment site.

43. A method as in claim 40, wherein the delivery system includes a distal end, and an expandable member near the distal end which comprises a balloon, and wherein the method further includes deploying the balloon and bringing the balloon surface into contact with the vessel wall.

44. A method as in claim 42, wherein the catheter further includes an expandable balloon structure, and an inflation lumen, and wherein the method further comprises inflating the expandable balloon structure by moving an inflation medium through the inflation lumen.

45. A method as in claim 42, wherein the catheter further includes an expandable balloon structure, and wherein the method further comprises inflating the expandable balloon structure so as to contact the adjacent vessel wall.

46. A method as in claim 42, wherein the enlarging agent comprises an etching agent, the catheter further includes an expandable balloon structure, which includes a porous surface, and an inflation lumen, and wherein the method further comprises delivering the etching agent through the inflation lumen into the expandable balloon structure and out of the porous surface thereof.

47. A method as in claim 40, wherein the enlarging agent comprises an etching agent, and wherein the method further comprises etching the endothelium by the etching agent in the localized areas of the treatment site, and creating channels thereby into the vessel wall.

48. A method as in claim 42, wherein the catheter further includes an expandable balloon structure, and wherein the method further comprises administering a solution containing the therapeutic agents through the expandable balloon structure into the created channels, trapping the therapeutic agents within the coronary vessel wall, for treating the vascular wall.

49. A method as in claim 43, wherein the system includes a radiant energy element, and wherein the method further includes coupling the balloon to the radiant energy element, and activating the radiant energy element to transmit energy through the radiant energy element.

50. A method as in claim 45, wherein the method further comprises tracking the system through the vasculature to the treatment site.

51. A method as in claim 48, wherein the enlarging agent comprises a hydrophilic acid agent, and the catheter further includes an expandable balloon structure, which includes a porous surface, and an inflation lumen, and wherein the method further comprises delivering the hydrophilic agent through the inflation lumen and the porous surface of the expandable balloon structure into the vessel wall.

52. A method as in claim 48, wherein the enlarging agent comprises a radiant energy element which comprises a radiant energy element which comprises a source of radiant energy, and wherein the method further includes coupling the system to the radiant energy element, and activating the radiant energy element to transmit radiant energy therethrough.

53. A method as in claim 40, wherein the delivery system is further able to deliver a therapeutic agent to the delivery site, so that the therapeutic agent will be delivered thereby into the enlarged migration channels at the treatment site, to treat the treatment site with the therapeutic agent, and wherein the method further comprises advancing the therapeutic agent into the enlarged migration channels at the treatment site, to treat the treatment site with the therapeutic agent.

54. A method as in claim 40, wherein the endothelium migration channels enlarged by the enlarging agent comprise endothelial cell gaps, and wherein enlarging in the method comprises enlarging endothelial cell gaps by the enlarging agent upon delivery of the enlarging agent to the treatment site.

55. A method as in claim 40, wherein the endothelium migration channels enlarged by the enlarging agent comprise internal elastic lamina gaps, and wherein enlarging in the method comprises enlarging internal elastic lamina gaps by the enlarging agent upon delivery of the enlarging agent to the treatment site.
